(19) United States
(12) Patent Application Publication
(10) Pub. No.: US 2025/0346850 A1
(43) Pub. Date: Nov. 13, 2025

(54) AUTOMATIC CULTURE DEVICE AND AUTOMATIC CULTURE SYSTEM USING MULTIPLE AUTOMATIC CULTURE DEVICES

(71) Applicant: Hitachi High-Tech Corporation, Tokyo (JP)

(72) Inventors: Shuhei HASHIBA, Tokyo (JP); Toyoshige KOBAYASHI, Tokyo (JP); Kensuke AMEMIYA, Tokyo (JP); Kunihiro FUJIKI, Tokyo (JP); Katsura NORIMATSU, Tokyo (JP); Yosuke INOSHITA, Tokyo (JP); Masakazu KAWANO, Tokyo (JP); Makoto ABE, Yokohama-shi (JP); Masaaki SUGIYAMA, Yokohama-shi (JP); Daisuke KANDA, Yokohama-shi (JP)

(21) Appl. No.: 19/051,365

(22) Filed: Feb. 12, 2025

(30) Foreign Application Priority Data
May 9, 2024 (JP) 2024-076595

(51) Int. Cl. C12M 1/36; C12M 1/00; C12M 3/00

(52) U.S. Cl. CPC C12M 41/48; C12M 23/22; C12M 23/34; C12M 23/38; C12M 23/44; C12M 23/58; C12M 29/00; C12M 41/14

(57) ABSTRACT

The automatic culture device includes an incubator capable of accommodating at least one cell culture vessel, and a flow path unit configured to supply gas and liquid necessary for cell culture to the cell culture vessel. The incubator and the flow path unit are arranged horizontally in parallel. The automatic culture device includes a front door capable of covering front surfaces of both the incubator and the flow path unit. The front door has a structure divided into upper and lower parts, the front door on an upper side can be lifted upward and includes a display unit capable of displaying a state of the automatic culture device and/or an operation unit capable of operating the automatic culture device, and the front door on a lower side can be opened by being pulled downward.

### CLAIM OF PRIORITY

The present application claims priority from Japanese Patent Application JP 2024-076595 filed on May 9, 2024, the content of which is hereby incorporated by reference into this application.

### TECHNICAL FIELD

The present invention relates to an automatic culture device for culturing cells or tissues, and an automatic culture system using a plurality of automatic culture devices.

### BACKGROUND ART

A medical treatment using regenerated human cells and cells with modified functions through gene transfer can treat diseases that have been difficult to cure so far. Therefore, the medical treatment is expected to be widely used.

The most common treatment target is a cancer, followed by diseases of various organs. This is considered to be because autologous transplants using the cells of patients have a low possibility of rejection and are in high demand from the perspective of improving patient quality of life (QOL).

Immune cells are the most common cell type targeted in cell therapy and occupy 40% or more of the total. The main target disease is cancer, and examples of specific cell types include immune cells such as T cells, NK cells, and NKT cells. In particular, the practical application of chimeric antigen receptor-T (CAR-T) cell therapy, in which T cells, which are immune cells, are extracted from a patient, processed by gene transfer into a form capable of attacking cancer cells, and then returned to the patient by injection, is currently at the forefront.

In a step of producing cells for transplantation, a biological sample taken from a patient or another person is separated and purified, and subjected to processing such as amplification and genetic introduction.

This step is performed in a cell processing center/cell processing facility (CPC/CPF) in accordance with a standard operating procedure (SOP) satisfying a law related to ensuring safety of regenerative medicine and the like (Act on the Safety of Regenerative Medicine) and an appropriate manufacturing standard (GMP: Good Manufacturing Practice or GCTP: Good Gene, Cellular, And Tissue-Based Products Manufacturing Practice) which is a standard for manufacturing management and quality management of medicines and the like. Therefore, the operation of the CPC/CPF requires significant costs and personnel with specialized culture techniques.

In addition, since the manufacturing step is mainly performed manually, there is a limit to an increase in the manufacturing amount. The low productivity and the high manufacturing costs of cells for cell therapy using them are impeding the widespread adoption of regenerative medicine, and there is a demand for automation of a culture operation that particularly requires labor and costs in a manufacturing step. It is possible to save labor, reduce costs, and achieve mass production due to automation of culture work.

The cell culture using the automatic culture device is contiguously performed for a long time, and depending on the steps, there are operations such as analysis and observation of samples and dispensing of a reagent, and it is necessary to sequentially take out the samples from the culture device at predetermined time intervals, replace the culture solution, and return the samples to the culture device again. PTL 1 describes an automatic culture device by which the efficiency of the operations is improved by shortening the movement time and distance of a culture vessel for analysis and observation of each sample, and the risk of contamination in a culture space from the outside of the device is reduced.

### CITATION LIST

### Patent Literature

PTL 1: JP2006-204187A

### SUMMARY OF INVENTION

### Technical Problem

In the automatic culture device, it is desirable that the conditions of the device can be set while checking the actual operation in the flow path and the display on a display unit. FIG. 1 of PTL 1 shows that an outer upper door 10a and an outer lower door 10b are provided, and a sample placed on the lower door 10b can be observed by an illumination 14 provided on a back surface of the upper door 10a. However, there is no description about setting of conditions of the device, and therefore, it is assumed that a computer provided separately from the culture device 1 is operated. In such a device, a flow path or the like in the culture device cannot be seen while operating the computer, and it is assumed that an efficient operation is difficult.

An object of the invention is to provide an automatic culture device which allows an operation unit to be operated while viewing a flow path or the like with an upper door opened, allows an actual operation in the flow path or a display of a display unit to be checked, and allows a power supply or a touch panel to be operated with a front door being in any open state or a closed state, and an automatic culture system using a plurality of automatic culture devices.

### Solution to Problem

A configuration of the invention for achieving the above object is as follows.

An automatic culture device includes an incubator configured to accommodate at least one cell culture vessel, and a flow path unit configured to supply gas and liquid necessary for cell culture to the cell culture vessel. The incubator and the flow path unit are arranged horizontally in parallel. The automatic culture device includes a front door configured to cover substantially entire front surfaces of both the incubator and the flow path unit. The front door has a structure divided into upper and lower parts. The front door on an upper side is openable by being lifted upward and includes a display unit configured to display a state of the automatic culture device and/or an operation unit configured to operate the automatic culture device, and the front door on a lower side is openable by being pulled downward.

### Advantageous Effects of Invention

The invention can provide an automatic culture device which allows an operation unit to be operated while viewing a flow path or the like with an upper door opened, allows an actual operation in the flow path or a display of a display unit to be checked, and allows a power supply or a touch panel to be operated with a front door being in any open state or a closed state, and an automatic culture system using a plurality of automatic culture devices.

### BRIEF DESCRIPTION OF DRAWINGS

FIG. 1 is a view showing a configuration of an automatic culture device.

FIG. 2 is a view showing a structure of an opening/closing door of the automatic culture device.

FIG. 3 is a view showing a structure of upper and lower doors of the automatic culture device.

FIG. 4 is a side view of the automatic culture device.

FIG. 5 is a view showing a structure of a side panel of the automatic culture device.

FIG. 6 is a view showing states of the opening/closing door when two automatic culture devices are stacked.

FIG. 7 is a view showing a relationship between the upper and lower doors and the internal bottle when the upper and lower doors of the automatic culture device are closed.

FIG. 8 is a perspective view of the automatic culture device in which a cut-out portion is provided in a side panel.

FIG. 9 is a view showing an indicator lamp provided at the top of the lower door of the automatic culture device.

FIG. 10 is a view showing a configuration of an automatic culture system in which a plurality of automatic culture devices are connected.

### DESCRIPTION OF EMBODIMENTS

Hereinafter, embodiments of the invention will be described with reference to the drawings. The following description shows a specific example of the content of the invention, and the invention is not limited to the description, and various changes and modifications can be made by those skilled in the art within the scope of the technical idea disclosed in the description.

In addition, in all the drawings for illustrating the invention, components having the same functions are denoted by the same reference signs, and repeated description thereof may be omitted.

### Embodiment 1

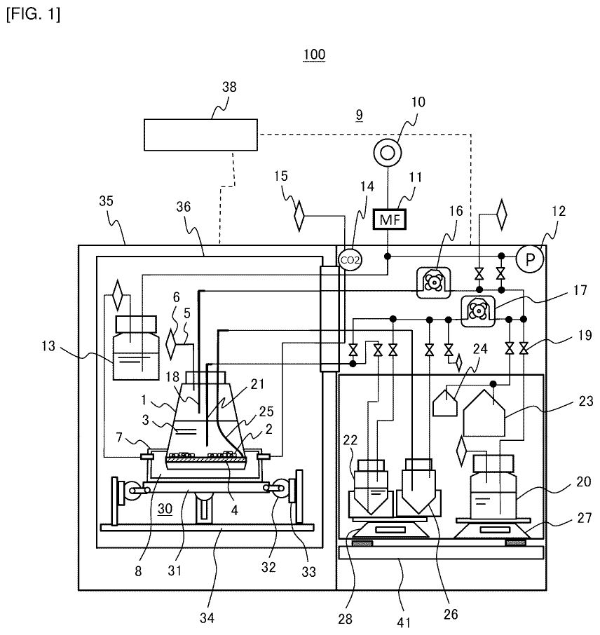

Components of an automatic culture device of the present embodiment which performs culture using a closed culture vessel will be described with reference to FIG. 1.

The automatic culture device 100 includes a culture vessel 1, a ventilation adapter 7, a gas supply unit 9, a pump for feeding a liquid or a gas, flow paths for connecting the components, valves for opening and closing the flow paths, a control unit 38 for controlling the gas supply unit, the pump, the valves, and the like, a swing mechanism 30 for swinging the culture vessel 1, and an incubator 35 as a temperature maintaining mechanism for accommodating the culture vessel, the ventilation adapter, the swing mechanism, and the like and performing temperature control.

The culture vessel 1 is a ventilation surface-equipped culture vessel having a ventilation surface 4 with a gas-permeable membrane arranged on a bottom surface, and cells 2 and a culture medium 3 are held and cultured therein. A pressure regulating pipe 5 and a vent filter 6 are connected to the culture vessel 1, and thus a gas inside the culture vessel can flow in and out, and the entry of bacteria and viruses from the outside is prevented.

The gas supply unit 9 includes a gas cylinder 10 in which a predetermined gas concentration is maintained, a gas flow rate control unit (mass flow controller: MF) 11, a pressure sensor 12, and a humidification bottle 13 serving as a humidification unit, and is connected upstream of the ventilation adapter 7. A CO₂ sensor 14 and a CO₂ gas vent filter 15 are connected downstream of the ventilation adapter 7, and the gas is discharged to the atmosphere outside the device. The CO₂ sensor 14 can monitor whether the gas replacement in the ventilation adapter is appropriately performed, and can be used to predict a culture state.

The feeding of a liquid or the feeding of a gas is performed by pumps 16 and 17, and a preferable pump is a tube pump that generates pressure by squeezing a rubber tube with a rotating roller. The pump 16 is configured to feed a culture medium to the culture vessel 1 and is connected to a liquid feeding pipe 18 of the culture vessel 1 by a tube. On the other hand, the pump 16 is connected to a supply pipe of a culture medium bottle 20 via a solenoid valve 19.

A solenoid valve is preferable as a valve for opening and closing a rubber tube constituting the flow path, and when electricity is applied, the solenoid valve is operated to open (the reverse operation is also possible) the rubber tube in the closed state of the rubber tube which is clamped by the valve operated by a spring force.

The pump 17 is configured to feed a cell suspension to the culture vessel 1 to perform cell seeding, or discharge the culture medium 3 from the culture vessel 1 to collect grown cells.

The pump 17 is connected to an aspirating tube 21 of the culture vessel 1 by a tube. On the other hand, the pump 17 is connected to a cell seeding bottle 22 via a solenoid valve 19 and is connected to a supernatant collection bag 23 and a supernatant analysis bag 24 via another solenoid valve 19.

The pump 17 is connected to a collection pipe 25 for cells in the culture vessel 1 by a tube, and on the other hand, is connected to a cell collection bottle 26 via a solenoid valve 19.

The weight of the culture medium bottle 20 is measured by a weight sensor 27, and the weight of the cell seeding bottle 22 and the weight of the cell collection bottle 26 are measured by a weight sensor 28.

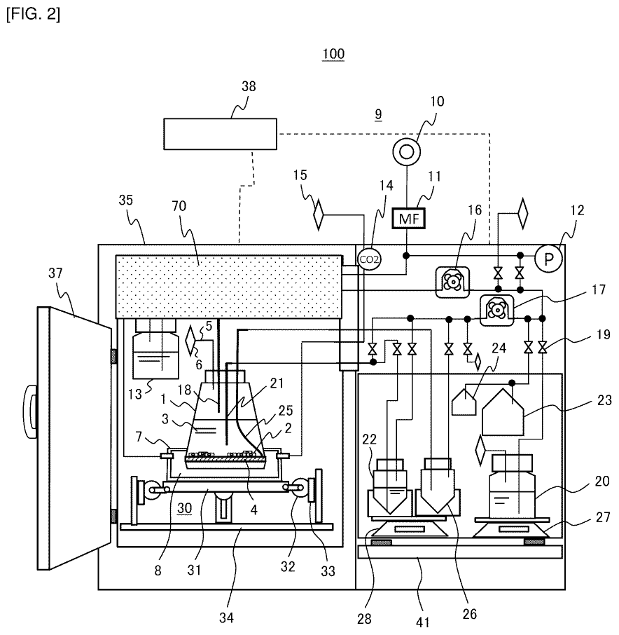

As shown in FIGS. 1 and 2, the culture medium bottle, the cell seeding bottle, the cell collection bottle, the supernatant collection bag, the supernatant analysis bag, the flow path connecting the components, the pump, the solenoid valve, and the weight sensor are provided in a fluid control unit 29 which is a device body outside the incubator 35.

The swing mechanism 30 includes a swing stage 31 for holding the ventilation adapter 7, link mechanisms 32 for supporting the swing stage from three directions, swing shafts 33 respectively connected to the link mechanisms 32, and a swing stage fixing mechanism 34 fixed to an inside of the incubator.

In a swing operation of the culture vessel, when control is performed such that the right swing shaft is lowered downward, the left swing shaft is raised upward simultaneously, and the central swing shaft is not moved, the swing stage moves with a tilt, and the culture vessel can be tilted. Next, when the motion of the left and right shafts is reversed, the culture vessel can be tilted in an opposite direction.

When the swing shafts are contiguously operated and the swing shaft is also moved in a depth direction of the paper, the culture vessel can be tilted back and forth, and therefore, the cells 2 and the culture medium 3 inside the culture vessel 1 can be stirred. Accordingly, not only the forward and backward movement and the leftward and rightward movement but also a three-dimensional swinging movement is enabled by a combination of the movement sequences.

The incubator 35 is an example of a temperature maintaining mechanism and is a so-called dry incubator including a constant temperature unit 36. The incubator 35 can house the culture vessel 1, the swing mechanism 30, and the humidification bottle 13, and can maintain the inside of the incubator at a temperature suitable for cell culture.

By adopting a small-sized dry incubator in place of a large-sized CO2 incubator and having a configuration capable of performing gas supply necessary for culture, the device can be downsized, and a plurality of devices can be simultaneously operated in a small space. The control unit 38 can control the operations of the gas supply unit 9, the pumps 16 and 17, and the solenoid valve 19, and the operation of the swing mechanism 30. By automatically controlling the mechanical elements at a predetermined timing, the cell suspension can be fed from the cell seeding bottle 22 to the culture vessel 1 during cell seeding, the gas humidified by the humidification bottle 13 can be supplied to the ventilation adapter 7 during gas replacement, the culture medium can be fed from the culture medium bottle 20 to the culture vessel 1 during culture medium addition, a culture medium can be supplied to the culture vessel 1 after the culture medium 3 in the culture vessel 1 is discharged to the supernatant collection bag 23 during culture medium replacement, and a part of the culture medium in the culture vessel can be fed to the supernatant analysis bag 24 during supernatant sampling.

During cell collection, after the culture medium in the culture vessel 1 is discharged to the supernatant collection bag 23, the cell suspension can be stirred by operating the swing mechanism 30 and then fed to the cell collection bottle 26.

In the present embodiment, the culture vessel 1 is attached to the ventilation adapter 7, and gas supply and replacement necessary for culture can be performed for the culture vessel 1, but a configuration may be adopted in which necessary gas supply and replacement can be performed for the housing of the incubator 35, and a plurality of culture vessels are placed in the incubator 35.

In FIG. 1, in order to show an internal mechanism of the automatic culture device, illustration of an opening/closing door and the like is omitted. A configuration of the opening/closing door and the like of the automatic culture device will be described with reference to FIG. 2. The automatic culture device includes an opening/closing door 37 for maintaining the internal temperature constant and a cover 70 (a portion filled with dots) for covering an internal temperature control device and the like. A section in which a flow path or the like on the right side of the automatic culture device is placed is referred to as a flow path unit. The flow path unit has a lower necessity of maintaining the temperature than the incubator, and is not provided with a door corresponding to the opening/closing door 37 in the present embodiment for the purpose of facilitating the operation of the valve and the like. However, an effect of preventing dust from entering from the outside can be expected by providing an opening/closing door, and therefore, the opening/closing door may be provided on the flow path unit side as necessary. In this case, it is preferable that the opening/closing door 37 is double doors so that opening and closing of both doors do not interfere with each other.

A transparent inner door that does not interfere with the opening/closing door 37 may be provided inside the opening/closing door 37. Accordingly, when the transparent inner door is closed and the opening/closing door 37 is opened, observation can be safely performed even when the swing mechanism 30 is operating. In addition, the transparent inner door is provided with a sensor, and thus can have an interlock function in which the swing mechanism 30 stops when the transparent inner door is opened during the operation of the swing mechanism 30.

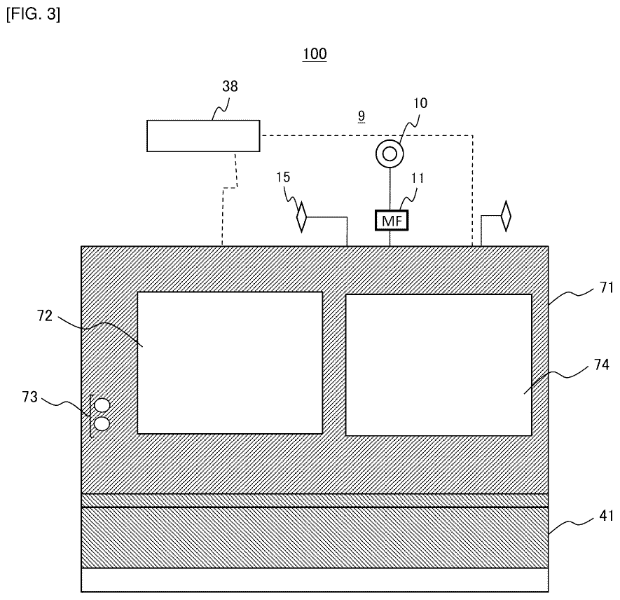

As shown in FIG. 3, an upper door 71 and a lower door 41 are further provided outside the opening/closing door 37 and the cover 70. When a manner of laterally opening the opening/closing door 37 and vertically opening the upper door 71 and the lower door 41 is used, the opening/closing door 37 of the incubator 35 can be prevented from interfering with the opening/closing operation of the upper door 71 and the lower door 41.

An observation window (also simply referred to as a “window”) 74 is provided on the right side of the upper door 71 so that the operation states of the flow path, a peristaltic pump, and the valve can be observed without opening the upper door 71. With this structure, if the flow path is damaged, the status inside the flow path unit can be checked without touching the internal liquid (including human cells (infection prevention)).

A display unit 72 implemented by a liquid crystal panel is provided on the left side of the upper door 71. The display unit 72 not only has a display function, but also functions as a touch panel. The display unit 72 may control the automatic culture device, or may simply function as a display unit and allow input from a wired/wireless connected mouse, keyboard, or the like. With such a configuration, an operation unit can be operated while viewing the flow path and the like in a state in which the upper door 71 opened. In addition, in a state in which the upper door 71 is closed, it is possible to check the operation state of the device by the display unit while observing the status of the flow path unit or to operate/control the device using the touch panel as necessary. When a size of the observation window 74 is adjusted to match a size of the display unit 72 having a standardized size, the device has a good appearance.

An operation unit 73 is provided on the left side of the display unit 72. When the operation unit 73 is provided with buttons that greatly affect operations of the automatic culture device, such as an ON/OFF switch and an emergency stop button of the device, the safety and usability are improved. The power supply and the touch panel can be operated regardless of whether the upper door 71 is open or closed.

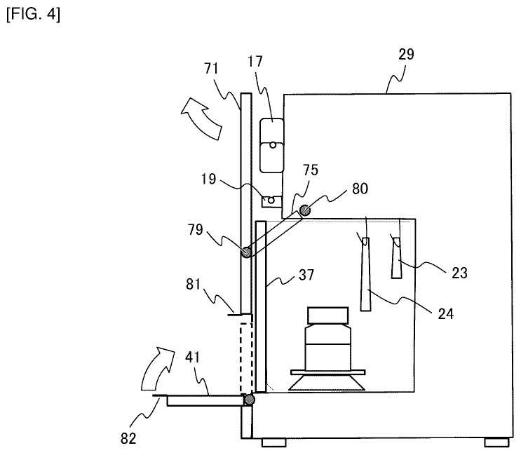

FIG. 4 shows the automatic culture device as seen from the side so that the structures of the upper door 71 and the lower door 41 can be seen. The upper door 71 is provided in front of the opening/closing door 37 and is supported by the housing of the automatic culture device by an arm 75 engaged with a rotation shaft 80 provided on the housing of the automatic culture device. The arm 75 is pivoted about the rotation shaft 80 as a rotation center by also providing a rotation shaft 79 on the upper door 71 side, and when the upper door 71 is lifted upward and opened, the upper door 71 can be opened while maintaining a substantially vertical state. When the upper door 71 is opened substantially vertically upward, the automatic culture device can be placed even when there is no room in a placement space (particularly, in front of the automatic culture device). When a device operator also opens the upper door 71, the upper door 71 is not opened to project to the front of the device, so that a distance in which the device operator moves backward or in a lateral direction in order to avoid the opening of the upper door 71 is reduced, and usability is improved.

The upper door 71 can be stopped at any position by adopting a free stop mechanism, which is a well-known technique using a frictional force, a gear, or the like, for the rotation shafts 79 and 80. Accordingly, it is possible to open the upper door 71 to the minimum necessary and perform an operation on a mechanism provided in an upper portion of the flow path unit, and it is possible to obtain an effect of preventing dust or the like from entering from the outside (the operation on the mechanism provided in a lower portion of the flow path unit can be performed by opening the lower door 41).

When the upper door 71 is opened, the operator can access the pump 17 and the solenoid valve 19 on a front surface of the automatic culture device. In addition, the lower door 41 becomes a shelf shape by being pulled down to the front side, and it is possible to temporarily place the culture vessel when replacing the culture vessel in the incubator, or to observe the internal state of the temporarily placed culture vessel.

In FIG. 4, the upper door 71 is supported by one arm 75 on the left and one arm 75 on the right, and one rotation shaft 80 provided in the housing of the automatic culture device is disposed on each side. Alternatively, if necessary, two arms 75 and two rotation shafts 80 may be provided at positions shifted in an up-down direction on each of the left and right sides. With such a configuration, even when the touch panel type display unit 72 or the operation unit 73 is operated in a state in which the upper door 71 is opened, the upper door 71 can be prevented from moving, and sufficient rigidity can be obtained even when the upper door 71 is heavy.

Further, an eaves-shaped protruding portion 81 is provided at the lowermost portion of the upper door 71. When the device operator opens and closes the upper door 71, the upper door 71 can be easily opened and closed by putting a hand on the protruding portion 81. In particular, when a plurality of automatic culture devices described below are vertically stacked and operated, the automatic culture device placed above is placed at a high position, and the protruding portion 81 is useful when a short operator opens and closes the upper door 71. An eaves-shaped protruding portion 82 is also provided at the uppermost portion of the lower door 41, and similarly, an effect in which the operator can easily open and close the lower door 41 is obtained.

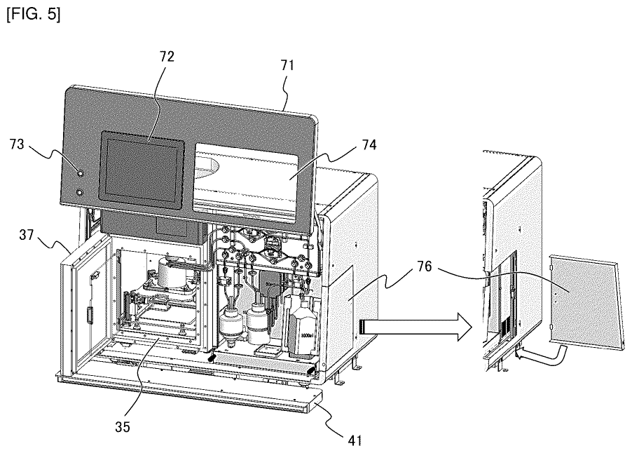

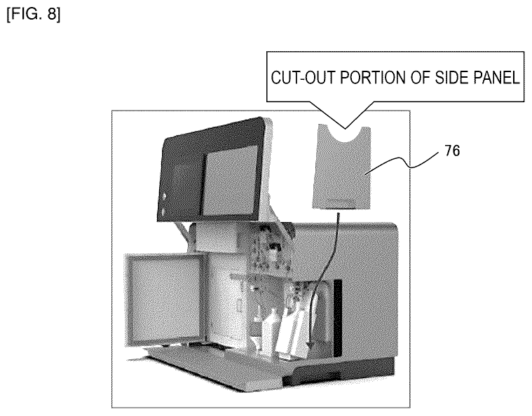

FIG. 5 is a perspective view of the automatic culture device. In order to facilitate access to the inside of the flow path unit arranged on the right side of the automatic culture device and facilitate maintenance, a detachable side panel 76 is provided (corresponding to “a structure in which a part of a housing covering a side surface is detachable” in claims). The culture medium bottle 20 and the like can be replaced by opening the side panel 76 even in a state in which the upper door 71 and the lower door 41 are closed, and therefore, usability for the operator is greatly improved. In addition, when the bottle is detached, workability of attachment and detachment of the bottle or the like is improved, and when the bottle is attached, effects such as less dust can be obtained, and appearance is also good. The side panel 76 may be provided with a cut-out portion (shown in FIG. 8) so that the tube of the flow path unit can be connected to the outside of the automatic culture device. Alternatively, the side panel 76 can be removed, and a tube or a bag can be used for function expansion of the automatic culture device such as connection to a device in a pre-process or a post-process of cell culture outside the automatic culture device or connection to an optional device for function expansion.

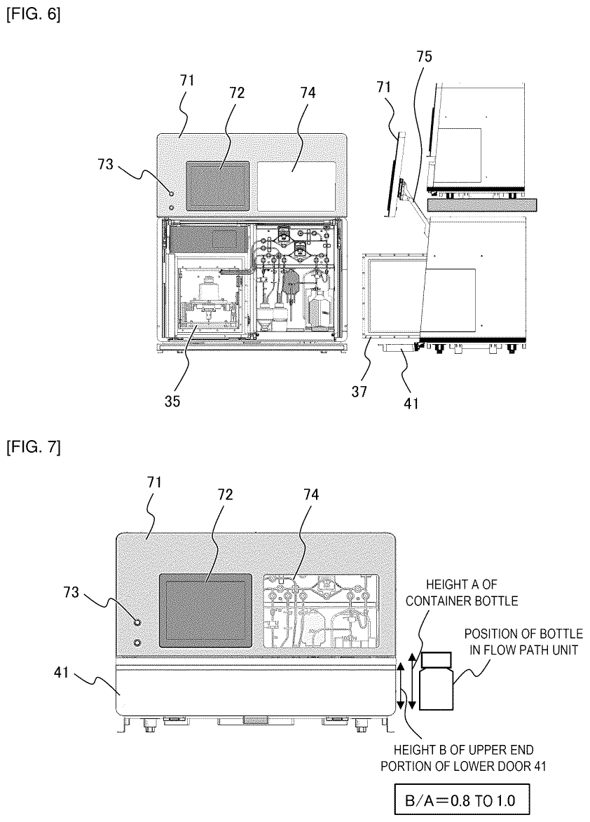

As shown in FIG. 6, the automatic culture devices can be vertically stacked to perform cell culture. When the automatic culture devices are stacked, even in a state in which the upper door 71 of a front door of an automatic culture device (a first automatic culture device) placed on the lower side is opened, the lower door 41 of a front door of an automatic culture device (a second automatic culture device) stacked above the first automatic culture device can be opened and closed (that is, a positional relationship in which the upper door 71 does not interfere with opening and closing of the lower door 41 is formed). With this structure, even in a state in which the upper door 71 of the first automatic culture device is opened, the lower door 41 of the second automatic culture device can be opened as necessary, and the culture vessel or the like can be taken out from the second automatic culture device and can be placed on an inner surface of the lower door 41 of the second automatic culture device which is opened to be in the form of a tray. Therefore, the usability for the operator is dramatically improved.

As can be seen from the left diagram of FIG. 6, when the automatic culture device is viewed from the front in a state in which the upper door 71 of the front door is opened to the maximum, the lowermost end of the upper door 71 is arranged so as not to overlap the flow path unit on the right side of the automatic culture device. With this configuration, the upper door 71 does not hinder the operation of the peristaltic pump, the valve, and the like arranged above the flow path unit, and the usability for the operator is improved.

When the device is viewed from the front with the lower door 41 closed, an upper end portion of the lower door 41 is configured to have a height that is 80% to 100% of a height of a vessel bottle such as the culture medium bottle 20 (B/A=0.8 to 1.0 in FIG. 7). With this configuration, it is possible to prevent the bottle from falling and to prevent an operation error or an error in erroneously opening an incubator door.

In addition, as shown in FIG. 8, an upper end surface of the side panel 76 may be provided with a cut-out portion (also referred to as a hole) for allowing a tube of a flow path system in the flow path unit to be exposed to the outside. With such a structure, when an optional device is added next to the automatic culture device and a tube is pulled out from a pipe of the flow path unit of the automatic culture device in the future, the tube can be easily passed, and the expandability is improved.

A shape of the cut-out portion may be, for example, a U-shape. In the case of the U-shaped hole, it is assumed that the tubes are bundled and taken out when there are a plurality of pulled-out tubes. However, by providing a plurality of cut-out portions (holes) having a size corresponding to each tube, it is not necessary to bundle the tubes, and it is also possible to adopt a configuration in which a necessary tube can be pulled out to the outside of the device.

In addition, the size of the upper door 71 changes depending on the size of the lower door 41, and therefore, it is preferable that the upper end surface of the lower door 41 is located below the uppermost portion of the bottle when the device is viewed from the front so that the bottle (vessel) placed in the flow path unit can be seen from the window 74 of the upper door 71. With this structure, it is possible to check whether there is an abnormality in the bottle and a flow path connection portion, which may cause a problem, without opening the door. In addition, a drive system (such as a peristaltic pump and a valve) in an upper portion can also be seen.

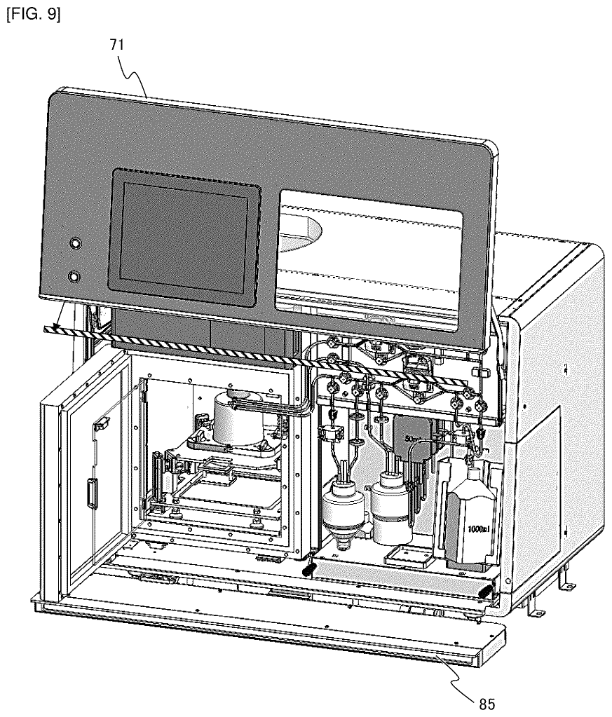

In addition, when an abnormality occurs in the device, it is preferable to provide a mechanism for quickly notifying the abnormality. However, in general, a flashing light (rotation light) is disposed above the device, and when an abnormality occurs, the flashing light is turned on together with a sound such as a buzzer. In FIG. 9, a horizontally long LED lamp capable of emitting all colors is disposed below the upper door 71. The LED lamp is lit in red when an abnormality occurs, and the LED lamp is lit in green when the LED lamp is in a normal state, so that an operator can quickly know the occurrence of the abnormality. The LED is disposed so that lighting of the LED can be seen from the front to the side surface. Further, the horizontally long LED lamp capable of emitting all colors may be disposed above the lower door 41.

FIG. 9 also shows a state in which the LED lamp is removed and moved downward (an area hatched with rectangular oblique lines indicates the removed LED lamp). When the LED lamp is provided at the lower end portion of the upper door 71, an effect is obtained in which the status display can be checked even in a state in which the upper door 71 is opened. In addition, the status can be displayed in an easy-to-understand manner without separately disposing a rotation light, and therefore, the user cost can be reduced. In addition, by providing a light distribution such that the LED lamp illuminates the inside of the flow path unit, it can be seen that normal cell culture is performed when the flow path unit is illuminated by the green LED, and it can be seen at a glance that an abnormality has occurred when the flow path unit is illuminated by the red LED. The color development can be adjusted according to demands of a user. From this point, it is also useful to provide the LED at the lower end portion of the upper door 71. In addition, the LED on the lower end portion of the upper door 71 can be used as a light that illuminates the hand during placement of the flow path, and by illuminating the inside of the flow path unit, dark is not easily visible, and workability can be improved.

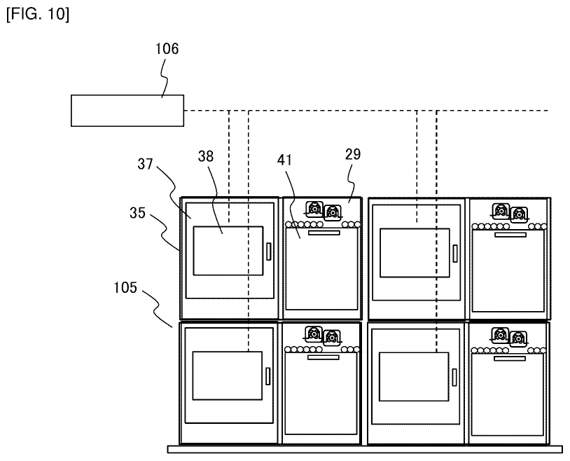

FIG. 10 shows an example of an automatic culture system in which a plurality of automatic culture devices are disposed, connected to a network, and controlled by a central management PC.

Four sets of automatic culture devices, each of which is a set of an incubator 35 and a fluid control unit 29, are disposed on a rack 105. In this embodiment, a rack on which four sets of devices are disposed, two sets on each of upper and lower stages, is shown. However, the number of placements per stage of the rack of this embodiment is any number, and it is easy to increase the number of integrated devices in a limited facility space of the CPC/CPF. From the viewpoint of accurate process management and sterile operation, it is also possible to fully automate all of the placement and removal processes of the flow path by a robot, and it is possible to manufacture the flow path beyond a working range that can be reached by a human hand.

Each control unit 38 is attached to a front surface of the opening/closing door 37 of the incubator 35 and is connected to a central control PC 106 by network equipment. The central control PC 106 has a function of monitoring a state of each of the automatic culture devices, a function of managing update and correction of an operation program, and a function of transmitting information when an operation abnormality of the device occurs or the operation ends. Furthermore, when the central control PC 106 is provided with a virtual display function, the control PC 106 can be provided with a function of the control unit 38. At this time, when any automatic culture device can be selected, the control PC 106 can operate and monitor a plurality of automatic culture devices.

By adopting the configuration described in the present embodiment, it is possible to provide an automatic culture device capable of simultaneously operating a plurality of devices in a space-saving manner, and an automatic culture system using a plurality of automatic culture devices.

### REFERENCE SIGNS LIST

1: culture vessel
2: cell
3: culture medium
4: ventilation surface
5: pressure regulating pipe
6: vent filter
7: ventilation adapter
8: gas space portion
9: gas supply unit
10: gas cylinder
11: flow rate control unit
12: pressure sensor
13: humidification bottle
14: CO₂ sensor
15: CO₂ gas vent filter
16, 17: pump
18: liquid feeding pipe
19: solenoid valve
20: culture medium bottle
21: aspirating tube
22: cell seeding bottle
23: supernatant collection bag
24: supernatant analysis bag
25: cell collection pipe
26: cell collection bottle
28: weight sensor
29: fluid control unit
30: swing mechanism

1. An automatic culture device comprising:
an incubator configured to accommodate at least one cell culture vessel; and
a flow path unit configured to supply gas and liquid necessary for cell culture to the cell culture vessel, wherein
the incubator and the flow path unit are arranged horizontally in parallel,
the automatic culture device includes a front door configured to cover substantially entire front surfaces of both the incubator and the flow path unit,
the front door has a structure divided into upper and lower parts,
the front door on an upper side is openable by being lifted upward and includes a display unit configured to display a state of the automatic culture device and/or an operation unit configured to operate the automatic culture device, and
the front door on a lower side is openable by being pulled downward.

2. The automatic culture device according to claim 1, wherein
an inner surface of the front door on the lower side is a tray on which an article is to be placed.

3. The automatic culture device according to claim 1, wherein
in a state in which the automatic culture devices are vertically stacked, cell culture is enabled in each of the automatic culture devices, and
even when an upper door of the front door of a first automatic culture device is opened, the upper door of the front door of the first automatic culture device does not interfere with opening and closing of a lower door of the front door of a second automatic culture device stacked on the first automatic culture device.

4. The automatic culture device according to claim 1, wherein
when the automatic culture device is viewed from a front in a state in which an upper door of the front door is opened to maximum, a lowermost end of the upper door does not overlap the flow path unit.

5. The automatic culture device according to claim 1, wherein
when the automatic culture device is viewed from a front in a state in which a lower door of the front door is closed, an upper end portion of the lower door is at a position having a height that is 80% to 100% of a height of a vessel bottle placed in the flow path unit.

6. The automatic culture device according to claim 1, wherein
a part of a housing configured to cover a side surface of the flow path unit opposite to the incubator is detachable.

7. The automatic culture device according to claim 6, wherein
a hole configured to allow a pipe to be pulled out from an inside of the automatic culture device to an outside of the automatic culture device is provided in a part of a surface of the detachable housing.

8. The automatic culture device according to claim 1, wherein
the incubator is provided with a laterally open door on an inner side of the front door on an incubator side.

9. The automatic culture device according to claim 1, wherein
a part of an upper door of the front door is provided with a window portion through which the flow path unit is observable from an outside of the upper door.

10. The automatic culture device according to claim 9, wherein
a size and a shape of the window portion are substantially same as a size and a shape of the display unit.

11. The automatic culture device according to claim 9, wherein
when the device is viewed from a front, an upper end surface of a lower door of the front door is positioned below an upper end portion of a vessel placed in the flow path unit such that the vessel is visible from the window portion.

12. The automatic culture device according to claim 1, wherein
an upper door of the front door is liftable upward to be held at any position.

13. The automatic culture device according to claim 1, wherein
an indicator lamp configured to display a state of the automatic culture device by changing a color is provided at a substantially lower end portion of an upper door of the front door.

14. The automatic culture device according to claim 13, wherein
the indicator lamp is configured to illuminate the flow path unit.

15. An automatic culture system comprising:
a plurality of the automatic culture devices according to claim 1; and
a centralized management computer configured to control, operate, and monitor the plurality of automatic culture devices.

16. The automatic culture system according to claim 15, wherein
the plurality of automatic culture devices are arranged vertically or horizontally in parallel.
